(19) United States
(12) Patent Application Publication
(10) Pub. No.: US 2014/0024971 A1
(43) Pub. Date: Jan. 23, 2014

(54) Assessment and cure of brain concussion and medical conditions by determining mobility

(71) Applicants: Frank E. Bunn, Thronhill (CA); Richard D. Adair, Waterloo (CA)

(72) Inventors: Frank E. Bunn, Thronhill (CA); Richard D. Adair, Waterloo (CA)

(21) Appl. No.: 13/550,930

(22) Filed: Jul. 17, 2012

(51) Int. Cl. A61B 5/103

(52) U.S. Cl. 600/595

(57) ABSTRACT

A mobility assessment determines the abnormalities and impairments in a subject's movements by administering mobility and mobility impairment active logic engine algorithms to the video data of the subject's movement. The determined abnormalities and impairments are administered to known norms for a particular test to determine if the abnormalities and impairments are normal or not. The determined abnormalities and impairments can be administered to known norms for brain concussions or injuries and for Multiple Sclerosis or Alzheimer's Dementia to classify whether or not the concussion, injury, Multiple Sclerosis, Alzheimer's Dementia condition exists and to determine the condition's phase and recovery rehabilitation progress. The results may be administered to determine a treatment regime to restore the subject's health thereby cure the condition.

### FIELD OF THE INVENTION

The present invention relates to systems and methods of determining and curing brain concussion and medical conditions, their affects, and determining recovery rehabilitation and monitoring its success, by assessing mobility of a subject.

### BACKGROUND OF THE INVENTION

It is recognised in the medical community that assessment of a subject's movement, generally referred to as mobility, may be used as an indicator of medical conditions, such as but not limited to diseases such as Multiple Sclerosis, Parkinson's, Dementia, Cerebral Palsy, Stroke, Heart Attack, as well as brain concussions and injury. A reduction in, or lack of, mobility, that is a mobility impairment, can carry an attendant risk of the existence of brain injury, brain concussion or Traumatic Brain Injury (TBI) which we will generally refer to as Concussion. It is also well known that brain concussions for subjects of all ages and particularly involving injury by falling or physical shock to the body especially to the head such as but not limited to sports injuries are one of the serious health problems often resulting in reduced mobility that could lead to long-term impairment or even death. The medical costs for treatment, recovery, and rehabilitation, of a brain concussion subject as well as costs to public and private health care systems from these concussions is devastating. In the cases of athletes, and especially those in professional sports, the financial costs due to lost playing time of highly paid athletes to related sports teams or employers are expensive and the medical expenses incurred by these athletes and by their teams can be very high.

To cure brain concussion and other medical conditions affecting the brain, and their effects of such health problems is the goal of the systems and methods revealed in this patent. The Wikipedia 2010 encyclopedia defines cure as the “restoration of health; recovery from disease” and “a method or course of medical treatment used to restore health”. The Collins English Dictionary—Complete and Unabridged, HarperCollins Publishers, 2003 defines cure as “a medicine or therapy that cures disease or relieves pain, curative, therapeutic, remedy treatment, intervention care provided to improve a situation”. Herein we will use the definition of our cure as the reduction, arrestment, remission, or reversal of the brain impairments of the subject reflected in their mobility impairments detected by the methods and systems of this patent and administering of the relationship rehabilitation procedures recommended by the said methods and systems to restore the subject's health.

It is widely understood in the medical field that the brain, whether a human or animal brain, in its operation, control, and thinking process is adaptable, changeable, able to learn new skills or relearn old skills lost due to accident, injury, illness, or disease. Dr. Norman Doidge, M.D. in his book The Brain that Changes Itself published in 2007 sites dozens of cases of patients recovering from losses of brain functions all of which illustrate what he calls the fundamental brain property of neuroplasticity. Through actual case histories of subject's, he demonstrates this neuroplasticity is: “neuro is for “neuron”, the nerve cells in our brains and nervous systems; and plastic is for “chageable, malleable, modifiable.” Dr. Doidge sites many examples of patients recovering mobility and lost memory from injury, illness, disease, stroke as well as from drug and radiation effects, all due to the brain being able to use other non-damaged cells to relearn skills and recover lost control. In short, the brain can learn or relearn lost skills and Dr. Doidge states that the use-it-or-loss-it axiom is true in brain recovery. Critical to this recovery process Dr. Doidge sites Dr. Michaeal Bernstein, M.D., Birmingham, Ala. USA., who developed a “constraint-induced movement” therapy of repetitive movements, actions or routines done for extended periods of time daily, as the plasticity-based treatment for patients who have lost control or function of a variety of skills such as movement of their limbs, or reasoning powers of their thinking, or vision or verbal deteriorations due to these brain impairments.

Through determination of mobility impairments of subjects by determining their mobility with the methods and systems of this patent and administering determined rehabilitation relationships for constrained-induced movements, exercises and procedural repetitions utilizing the neuroplasticity of the subject's brain we can cure these patients by helping them to recover lost skills, movements, memory thereby restoring their health to the maximum possible for each subject. Each subject and their individual health problems will differ from patient to patient as will the degree of cure that is possible for that subject.

Sports injuries for young athletes are particularly worrisome health problems. Dr. Casey Taber, from the San Antonio Orthopaedics Group, in an Oct. 6, 2010 radio interview, confirms that the estimated 3,500,000 hospital visits per year due to child sport injuries, as published by the U.S. Centers for Disease Control, AOSSM, (http:/www/cdc/gov), “are 5 times higher than 10 years ago, and this has been increasing steadily over ten years”. The U.S. Centers for Disease Control and Prevention states that from 2009 Emergency Department visit data, 248,418 concussions were recorded related to sports and recreation activities. The Cleveland Clinic Sports Health Center in Ohio estimates that annually in the US, high school athletes suffer 2,000,000 injuries; have 500,000 doctor visits; and have 30,000 hospital visits due to sports activities. The Clinic has recently announced a “New Campaign STOP Sports Injuries in Kids”. The Clinic's Concussion Center states: “US athletes at all levels of competition, suffer more than 1,000,000 concussions each year. Most concussions will resolve within 7-10 days”. But he notes that: “Effects of a concussion can last several months, and rarely, may have long-lasting effects . . . ”.

Another demographic showing rising cases of concussion relates to the armed forces. The Cypress Newspaper (Cypress Texas) in a Jul. 7, 2001 article estimates that 300,000 Veterans have sustained concussion from the Iraq and Afghanistan campaigns. Dr. Drew Helmer, Associate Dir. of Research—Prime Care is actively recruiting participants for several clinical research studies investigating these new cases of concussions in veterans at The Michael E. DeBakey VA Medical Center. In 2009 the Center was awarded $5 million for research focusing on mild to moderate concussion with the Neurorehabilitation: Neurons to Networks Center of Excellence focusing on mild concussion. “Most Veterans with mild TBI recover fully; but some have longer lasting problems that can interfere with their ability to work or get along with their friends and family,” said Dr. Helmer, who is also an assistant professor of Medicine at Baylor College of Medicine and director of Recruitment and Retention. Dr. Robin Green of The Toronto (Ontario) Rehabilitation Institute and related Canadian Sports Concussion Project at the Krembil Neuroscience Centre, Toronto Western Hospital notes: “There is increased awareness of and concern about TBI due to the large number of such injuries being sustained by soldiers in Iraq and Afghanistan. Some of our findings are highly relevant to those individuals.” For example, one study shows the inadequacies of conventional diagnostic approaches for people with the milder, yet still debilitating, brain injuries of the kind many soldiers are sustaining

Vicky Scott, British Columbia Injury Research & Prevention Unit, Ministry of Health, Office for Injury Prevention, Victoria BC, Canada, et al., in 2008 published results of an exhaustive review of published studies that test the validity and reliability of fall-risk assessment tools, titled “Multi-factorial and functional mobility assessment tools for fall risk among older adults in community, home-support, long-term and acute-care settings”.

The Cleveland Clinic Sports Health Center in Ohio defines: “A concussion is an injury to the brain that results in temporary loss of normal brain function. It usually is caused by a blow to the head but can also be caused by whiplash injury of the head and neck. Cuts or bruises may be present on the head or face, but in many cases, there are no signs of trauma. Concussions do not necessarily involve loss of consciousness.” “Even mild concussions should not be taken lightly”. “A concussion can affect concentration, memory, judgement, reflexes, speech, balance, and muscle coordination.”

Concentration, reflexes, balance and muscle coordination all can affect a subject's mobility. Assessing the degree of the subject's mobility or mobility impairment provides a new and significant tool for detection and monitoring of the effects of concussions, tracking success of the treatment for concussions and for the determination and the implementation of procedures, practices, and aids to improve the subject's mobility, activity and their recovery and return to a healthy condition and regular life activities.

Many patents have taught methods and instrumented apparatuses related to measuring parameters for mobility, stability and walking, and devising systems to aid, correct and rehabilitate movement of subjects as related to their risk of falling. Nashner, in 1997 U.S. Pat. No. 5,623,944 and again in 2000 U.S. Pat. No. 6,010,465, teaches the use of mechanical treadmills instrumented with sensors connected to computers to measure a subject's walking gait. Sol, in 2001 U.S. Pat. No. 6,231,527, teaches the use of mechanical treadmills instrumented with sensors, plus the addition of several video cameras and mirrors, producing data related to weight-bearing forces on a subject's feet while walking in instrumented shoes as a method for analyzing walking difficulties and determining orthotic solutions. Adrezin, in 1996 U.S. Pat. No. 5,511,571, teaches using mechanical walking aids such as walkers, canes or crutches wherein the actual aids are themselves instrumented with sensors to measure force loads in those aids from which to measure the gait of a walking subject.

Many patents have taught methods and instrumented subjects related to measuring parameters for a subject's body mobility, stability and walking, and devising systems to aid, correct and rehabilitate movement of those subjects as related to their risk of falling. Ng, in 1998 U.S. Pat. No. 5,807,283, teaches use of a magnetic sensor strapped to the leg of a subject, plus additional instrumentation strapped to the subject's other leg or to a specialized shoe worn by the subject, from which data are transmitted to receiving and systems to measure the speed and gait of the subject. Weir, in 1998 U.S. Pat. No. 5,831,937, teaches the use of a transponder worn about the middle of the subject's centre of mass, which transmits infrared and ultrasound pulses to receiver and computer systems, from which data gait, speed, cadence, step time and step length are determined for assessment of gait pathologies. Allum, in 1999 U.S. Pat. No. 5,919,149, teaches use of angular velocity transducers attached to the upper body of a subject, to detect the movement not of a subject's feet but of the subject's body swaying in angular position and velocity, plus specialized eyewear, from which data an operator may interpret balance or gait disorders.

Many patents have taught methods and instrumented subjects related to measuring parameters of a subject's feet movement relating to the subject's walking gait. Takiguchi, in 2007 U.S. Pat. No. 7,172,563, teaches using a microphone attached to a subject's body for picking up low frequency sounds from their feet, and an analyzer of the sounds transmitted through the subject's body while walking, from which gait characteristics of that specific subject can be determined. Hubbard, in 2002 U.S. Pat. No. 6,360,597, teaches the use of force-sensing sensors installed in a shoe insert worn by a subject, from which sensor electrical output data are analyzed for of gait of a walking subject. Haselhurst, in 2007 U.S. Pat. No. 7,191,644, teaches the use of a pressure sensor and personal annunciator system installed in a shoe insole worn by a subject having difficulty walking, with which the system can tell the subject when the foot is contacting the floor, as a gait assistive device. Au, in 1989 U.S. Pat. No. 4,813,436, teaches the use of pressure sensors installed in the shoes or in shoe inserts worn by a subject, for measuring the subject's gait while walking, plus the use of video signals from two video cameras recording the motion of the subject who is wearing strategically placed visible markers such as on knees, elbows, and hips such that these data, along with the gait measurements, are presented to a practitioner to judge the subject's walking gait and, by overlaying these data on the video and gait of a “normal” subject, allows comparisons to be made.

Many patents have taught diagnostic tests administered to a subject such as in U.S. Pat. No. 6,383,150, in which a subject's balance disorder is tested by balance testing machines whose data are networked to physicians and clinical experts for their subjective interpretation of these balance diagnostic data. U.S. Pat. No. 5,980,429 teaches a system and method for monitoring training programs by a “Trained program prescriber” making a subjective interpretation. U.S. Pat. No. 7,720,530 teaches a field-deployable concussion detector for on-site diagnosis to determine concussion requiring the invasive placement of an electrode set on the subject plus using of a hand-held means for processing brain electrical signals. U.S. Pat. No. 7,046,151 teaches even more invasive systems of a body suit and interactive limb covers for entertainment games and feedback system to the subject's limbs. USPTO application 20110270135 teaches invasive system of wearable display glasses with multiple passive controllers measuring the subject's motion related to the glasses display seen by the subject as augmented reality to determine if such motions could cause injury or reduced performance. USPTO patent application 201200004034 teaches invasive physiologically modulating videogames or simulations and motion sensing input devices, for the subject to monitor the motion to enhance personal psychophysiological improvement. USPTO application 20090000377 teaches an invasive system requiring the subject to wear a body mounted impact device and transmitter and reader for measuring potential brain impact severity.

Dr. Ann McKee of Bedford Veteran's Clinic, US Dept. of Veteran's Affairs notes that typically athletes and sports players often hide any symptoms or information or possible occurrences of concussions in order to protect their jobs or positions in the sport or activity. Researchers at Georgia Tech and Emory University, Atlanta Ga., Readiness to Play Athletic Trainers and Coaches note: “An athlete who appears to be “fine” may not really be ready to get back onto the playing field.” “However, sometimes the signs of a concussion are very subtle.” If results of verbal tests are uncertain, further assessment is needed. “Typically, that requires about one to two hours in a quiet room.” These researchers have devised a Display Enhanced Testing for Concussions and mild Traumatic brain injury system composed of a head-mounted display unit, earmuffs, an input “joystick” and portable computer. The athlete responds to computer commands appearing on an LCD screen by using the joystick. They suggest players could use the system before injury to establish a baseline response and then after suffering an injury suspected as brain concussion from which computer software may be able to pick out subtle differences indicating brain injury.

In 2004 the Riddell football helmet manufacturer announced the launch of Riddell Sideline Response System, a new technology that combines a real-time, on-field head impact telemetry system (HIT System), team management software, and cognitive testing to provide a new standard of care for the athlete. The Minneapolis, Mimm., Star Tribune newspaper article, Aug. 9, 2007, notes the University of Minnesota Golden Gophers football team has used this 6-sensor helmet to measure G-forces of hits that the player sustains to the head and a transmitter sends an alert to a sideline computer.

However, specifically related to concussion whether from falls, impact, sports, accidents or occupational circumstances, Dr. Alexander Dromerick of the MedStar National Rehabilitation Hospital in Washington DC states: “Athletes that continue to play after an injury can put themselves at risk for more serious or fatal injury.” Dr. Dromerick is leading a clinical trial of a possible concussion screening tool using a computerized constant-grip and pointing “joystick” to assess the pointing and steadiness of grip of subjects that may be related to effects of concussion.

The problem with all of the above methods is that they are invasive to the subject, are conducted in artificial testing environments, and that they present only data which subsequently require a subjective interpretation of a skilled practitioner to interpret these data and draw conclusions as to the mobility of the subject, and in some cases to estimate the subject's risk of falling. None of these methods can obtain an objective assessment of the mobility of a subject as an indication of possible brain concussion. Dr. Dromerick more emphatically states: “Currently, there is no good method that can quickly detect brain injury or concussion.”

Where the subject is a professional athlete or a person active in sports, especially subjects under the age of 19, their work and play environment raises to high the risk of concussion. It is well known that these subjects are highly vulnerable to impact, shaking and shock of the brain and that such effects to the brain often cause the devastating effects of concussion to the subject, affecting their families, their employers, health insurers, lost time and activities and work, significant medical expenses and possible long term deterioration of well-being and quality of life. The known techniques for assessing such risks do not lend themselves to such an environment where a large population has to be monitored whether on a per injury occurrence basis or on a continuous basis.

It is therefore an object of the present invention to provide a system, method and apparatus in which the above disadvantages are obviated or mitigated.

### SUMMARY OF THE INVENTION

In general terms, the present invention provides a system for assessing the mobility of a subject, said system comprising a motion sensor or sensors to observe movement of a subject and generate a data stream representative of such movement, an active logic engine to determine abnormalities in such motion and determine relationships of said abnormalities to at least one known norm and an allocator administered upon a said active logic engine to determine whether said abnormalities are within said known norm.

In a further aspect, the invention provides a method of assessing mobility of a subject comprising the steps of recording motion of said subject, administering fuzzy logic algorithms with said active logic engine, on said motions to determine assessments of mobility and for determination of abnormalities of such movement, determining relationships of said abnormalities to known norms, and determining whether said abnormalities are within a known norm or range of known norms.

In a further aspect, the invention provides methods and systems of administering an allocator on said active logic engine to determine if said abnormalities are within a known norm or range of known norms whereby to determine the possible existence of concussion or brain injury and determine at what stage is the said concussion or injury, and determine relationships with known rehabilitation procedures and determine the administering of the relationship rehabilitation procedures recommended by the said methods and systems to restore the subject's health and cure the said brain concussion or injury.

### BRIEF DESCRIPTION OF THE DRAWINGS

Embodiments of the invention will now be described by way of example only with reference to the accompanying drawings in which:

FIG. 1 is a representation of a 2-D movement mobility assessment of a subject.

FIG. 2 is a schematic representation of a movement mobility assessment of the subject.

FIG. 3 is a schematic representation of a further functional test assessment process.

FIG. 4 is a schematic representation of deviation from “normal” movement by a subject.

FIG. 5 is a schematic representation of a further deviation from “normal” movement by a subject.

FIG. 6 is a schematic representation of “Normal” movement by two subjects and deviation from “Normal” by one subject in a hallway environment.

FIG. 7 is a block diagram of the computer decision-tree structure for assessment of the mobility of a subject potentially showing effects of brain concussion for a Sit—Stand—Turn—Sit, movement.

FIG. 8 is a block diagram of the computer decision-tree structure for assessment of the mobility of a subject potentially showing effects of concussion of a subject for a Walk-Slow—Negotiate—Walk-Fast, movement.

FIG. 9 shows the equations for computation of the Mobility Condition (M) and for the Mobility Coefficient (AM).

FIG. 10 is a block diagram of the computer decision architecture for assessment of mobility of a subject potentially showing effects of concussion of a subject.

FIG. 11 is a block diagram of the personal Assessor decision architecture with computer assistance for assessment of mobility of a subject potentially showing effects of concussion of a subject.

FIG. 12 is a schematic representation of stereoscopic 3-D observation of a subject in this example rising from a chair.

FIG. 13 is a block diagram of the logic decision architecture for the Basic Mobility Assessment segment of the two: Basic; and Advance; segments of the expert system.

FIG. 14 is a block diagram of the logic decision architecture for the Advanced Mobility Assessment segment of the two: Basic; and Advance; segments of the expert system.

### DETAILED DESCRIPTION OF THE INVENTION

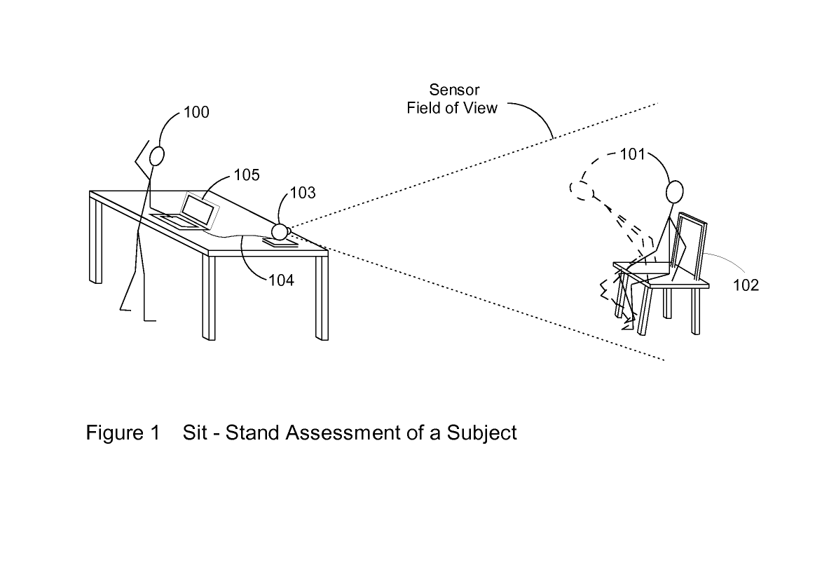

Prior to describing the system and its function in assessing mobility of a subject potentially showing effects of concussion, a number of the typical assessment environments will be described to provide context to the operation of the system. Referring therefore to FIG. 1, an expert system apparatus is used within a typical professional office environment for observing and video-recording the movements of a subject, (101). The system includes a computer (105) that implements an active logic neural networks decision engine, to administer the active logic engine algorithms to video data obtained from a motion sensor (103). The motion sensor (103) may be a camera operating in one or more of the visible, or infrared, or ultraviolet spectrum, an acoustic image capturing device or location sensors such as GPS positioning devices or RF motion/ location devices that generate information from which the movement of the subject may be determined. For convenience they will be collectively referred to as a camera. The data are subjected to administration of active logic engine algorithms and tests to enable an expert system to determine if the movement that has been observed is an abnormal condition, that is, one that departs from an expected or desired motion and commonly referred to as a mobility impairment condition. The system utilises that information to assess a particular condition, such as presence of a brain concussion. In FIG. 1, a subject (101, solid lines) sitting in a chair (102) is being observed by a camera (103), connected via wire (104) to the computer (105) being operated by a test facilitator (100). The camera (103) could also be connected to the computer (105) by one of the many available wireless communications devices. The test conducted requires the subject to arise from the chair. The camera (103) detects the motion of the subject (101) and transfers the data representing the motion to the computer (105) for further processing.

As an example, say the subject takes two attempts to rise from the chair (101, dotted lines). The camera (103) captures the movement of the subject (101) in a time dependant manner and transfers the data to the computer (105). The expert system embedded in the computer (105) operates on and administers the active logic engine algorithms to the data from the camera (103) and may prompt an observer to provide further inputs. This can be done in real time during the live observation process or operating off-line with the recorded video data following the observations.

As will be described more fully below, the expert system administers the new and uniquely developed stagger movement algorithms being revealed herein, which will be referred to as Mobility algorithms to determine abnormal movement and applies this information and additional input to provide the criteria required to apply the standardized test criteria, e.g. the Tinetti test parameters. In the example provided, the two attempts to rise are determined as a mobility impairment condition and these determinations indicate that the subject has a significant impairment for that movement.

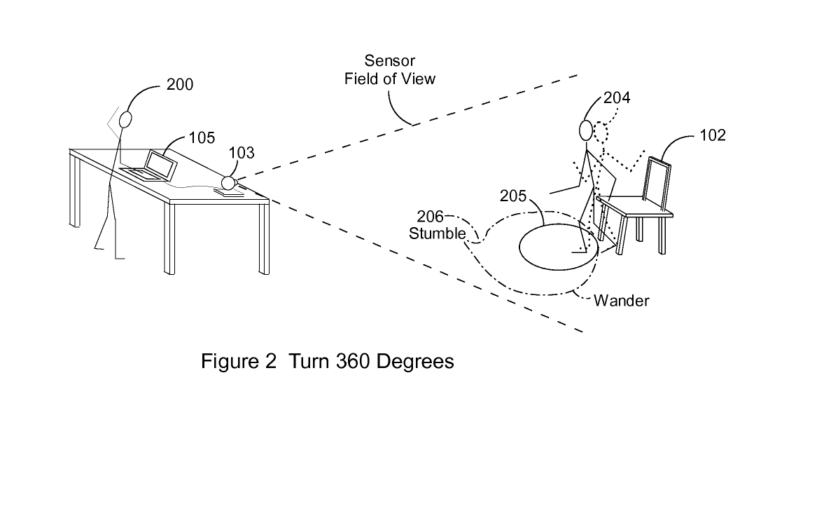

FIG. 2 shows a typical functional test assessment process and decision computations for a subject (201) to complete actions to rise from a chair (102), stand still, then turn around 360 degrees. The test facilitator (200) asks the subject, once the subject has risen from sitting in a chair (102), to stand still for assessing steadiness without wobbling or swaying, The computer (105) and the camera (103) capture the video data to record the movement indicated at (204), where solid lines stick-person subject and dotted lines stick-person subject indicate change of position over time to indicate that the subject is wobbling. In this test example, the expert system, administering the algorithms in real time or offline, may determine the wobble or swaying as being a mobility impairment condition. These determinations are provided to the selected established test procedures and risk scoring, and, depending on the cumulative results, the expert system may decide the subject has a significant impairment for that movement (204). The expert system also determines the level of mobility of the subject's actions while standing (204), wherein wobbling, swaying or stumbling is detected, recorded and scored.

Continuing with this example, the facilitator then asks the subject to turn 360 degrees along the path (205), for which the solid line indicates the expected circular track for normal turning. The expert system observes the actual movement (206) indicated by the dotted line and administers active logic engine algorithms to the sensor data to determine the wandering and stumbling as being a mobility impairment condition. This is input into established test procedures and mobility scoring to determine if the subject has significant mobility impairment for that movement.

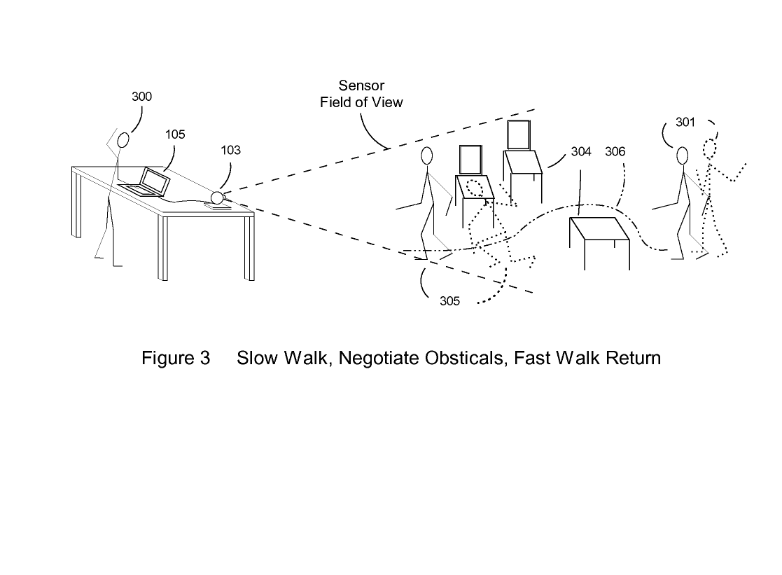

FIG. 3 shows a typical functional test assessment process and decision computations for a subject to complete actions from being in a standing position to then walk slowly forward while negotiating obstacles as a test of agility, vision, and mobility related to the subject's possible condition of brain concussion. The test facilitator (300) asks the subject (301), once the subject is standing still to walk forward, while the actions are captured with the computer (105) and the camera (103). The solid lines stick-person (301) subject and dotted lines stick-person subject indicate the subject is hesitating to start walking and may indicate possible cognitive problems. As the subject negotiates obstacles (304) any bumping or stumbling near them could indicate vision or agility problems related to concussion. Then in walking out from where the obstacles were arranged, the departure from the path (305) of the solid lines stick-person subject and dotted lines stick-person subject indicate that the subject is having some mobility impairment and recovers to continue walking

The expert system operating and analyzing the sensor data evaluates the movement to determine mobility impairment conditions, prompts for further input as needed according to the selected standardized test and determines whether the subject has significant mobility impairment for that movement. Similarly when test facilitator (300) asks the subject to retrace the path (306) back to the starting position but at a slightly faster pace, the corresponding observations and decisions can be made, as well as determining timing the difference between slow and fast walking, allowing the expert system to make further decisions on the mobility of the subject and the subject's potential of having concussion.

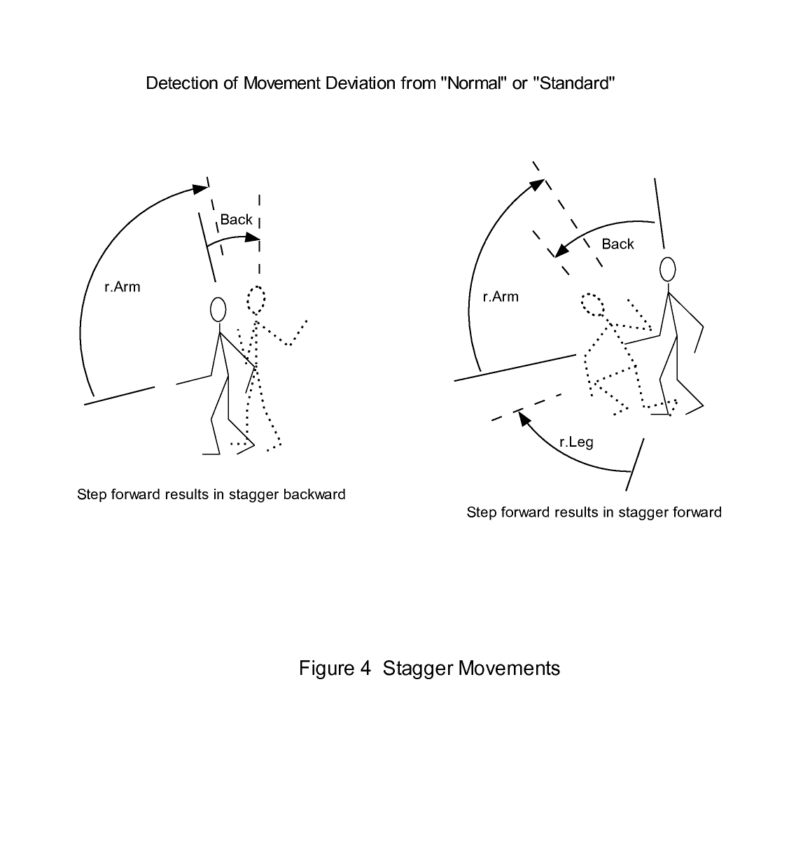

There are many actions that can be used to observe and assess mobility, the occurrence of mobility impairment and conditions of a subject and the potential of having concussion for that subject being assessed. FIG. 4 illustrates examples of two movements of a subject which would normally be determined by a mobility assessment algorithm to deviate from expected “Normal” or “Standard” movement. “Normal” means movement that has been previously observed and recorded in databases for this subject and is accepted as a base level of mobility for this subject. “Personal” can also be called as defined for a specific subject but can include a variety of levels of mobility for that subject possibly but not limited to being the history of that subject's mobility over time and health condition. “Standard” means movement that has been observed and recorded in databases of typical movements for subjects of similar age, sex, health, and mobility and is accepted as a base level of mobility for any similar subject.

The active logic engine algorithmic means revealed in this invention provides the algorithms to automatically administer them to the input video data from a multiplicity of sensors and video cameras, said algorithm means functioning as an administrator, to conduct detection determinations, and information extraction administrations, from which to assess the likelihood of the existence of brain concussion for a subject. This is accomplished by administering active logic engine algorithms to video data, to develop electronic or mathematical signatures of said functioning as an allocator to determine whether the output of said signatures is within known norms of the movement of “Personal”, and/or, “Normal” and deviations therefrom for movement of subjects which signatures are stored in the system in related databases. Then, deriving similar signatures of subjects to be assessed as to Mobility Impairment, the active logic engine algorithms determine the deviation of these signatures from the “Normal” signatures to make the decisions as to infer levels of impairment, and if it does exist, to decide whether the movement indicates concussion and if so required, it informs the appropriate personnel or systems. Similarly, determinations of the deviation of subject movements could result from medical emergencies such as heart attack, or seizure that also require healthcare personnel assessment in responding to the subject in question for which appropriate medical actions can be taken.

This invention teaches algorithms for administration by an expert system to data observed by a camera system from which administration to automatically determine the mobility of a subject. Herein, the new digital camera systems give rise to the ease of development and enabling of new computer applications such as the algorithmic means of this patent. Hundreds of hours of video of subjects have resulted in development of the algorithms revealed in this patent for the detection and assessment of the mobility of a subject. These algorithms permit determination of physical presence of conditions such as staggering and mobility impairment and physical conditions of the subjects being observed through administering the algorithms to the data from such sensors and systems and using active logic engine, and to conduct determinations of the potential presence of mobility impairment of the observed subject and potential existence of brain concussion.

The administration of the algorithmic system means using the active logic engine, can implement unique determinations and subsequent reporting means for mobility impairment and potential concussion. The camera system can record a subject and administering the algorithmic system can determine the mobility and can determine the conditions of the subject's face, eyes, pupil of the eye, walking gait and general appearance of the subject. Later, such observations of the subject will determine the changes in the subject's condition, appearance, and behaviour as it correlates to the earlier determinations and in real time determine any deviations that could relate to mobility impairment and possible existence of concussion or medical health condition as determined by the active logic engine algorithmic means. However, if the algorithm administration system means through access to related databases has access to medical and health information and database of related mobility impairment signatures of the subject, the active logic engine processor may be able to determine if the subject being observed is in fact having a health problem such as but not limited to heart attack, stroke, diabetic coma, epileptic seizure or brain related diseases such as but not limited to Multiple Sclerosis, Parkinson's, Dementia, Cerebral Palsy, or brain concussion, and any of which could be needing immediate medical assistance.

Using active logic engine algorithm means, integrated computer circuitry, and these data from a multiplicity of sensor systems, administration of the algorithms revealed herein were developed to isolate individual subjects relative to the background environment and to represent the image of the subject's movement in each video frame and the subject's appearance utilizing multiple control points superimposed on the subject's image. Overlaying a grid on the subject image, segmented image algorithms were created to represent the movement of the subject over selectable time slices. Administration of algorithms were created to mathematically represent these movements in vectored time space with which the algorithms were trained using the multitude of video data to determine “normal” mobility and deviations of same suggesting impaired mobility. The observations of images of movements of subjects can then be vectorized and matrix representations can be determined to estimate the degree of excursion from normal the subject's movement appears. Laboratory calibrations permit the estimate of the levels of mobility impairment of the subject. The administration of the algorithms can determine the levels of mobility impairment of subjects under surveillance in an observed real world environment utilizing the calibration and training of the algorithms with controlled laboratory data.

In a unique and preferred embodiment of this invention, we incorporate the administration of algorithms for automated determination of the facial conditions of the subject being observed. Conditions such as skin coloration such as flushed, or conditions such as sweating, or such as reddening of the eyes, or dilation of the eye pupil or pupils, or closing of the eyes, could be related to and thus can be used as in the determination of mobility impairment which possibly could be related to brain concussion. In cases where the environment may affect the subject's conditions the subject can be removed from the affecting environment for further observation and the algorithms can also be adapted to account for these affects. We reveal here algorithmic active logic engine system means to mathematically represent these observations and being a component of the active logic engine means determining mobility impairment and the allocator means operating on said active logic engine to determine the resulting mobility impairment and related possible brain concussion of the subject observed.

In the impaired mobility determined as a stagger back example in FIG. 4, the subject in attempting to step forward (solid line stick figure), actually staggers backward (dashed line stick figure) in which the major motions of the subject's back and right arm would be determined by the mobility impairment assessment algorithm to deviate from expected for either the “Normal” or “Standard” movement. In the stagger forward example, the subject in attempting to step forward (solid line stick figure), actually staggers forward (dashed line stick figure) in which the major motions of the subject's back and right arm and left leg would be determined by the mobility impairment algorithm to deviate from expected for either the “Normal” or “Standard” movement.

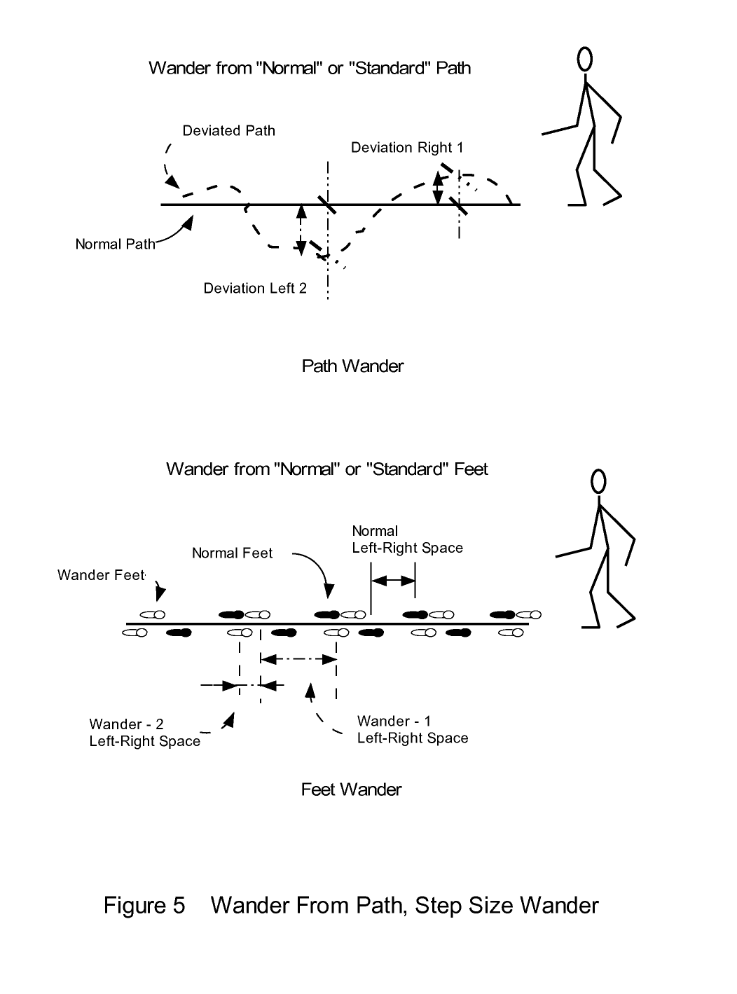

FIG. 5 illustrates movements of a subject's feet in which the subject's walking path wanders from a “Normal” or “Standard” path for the subject's feet indicated by a Deviation Right (1) and a Deviation Left (2) which would be determined by the stagger algorithm to deviate from expected for either the “Normal” or “Standard” movement. Further, FIG. 5 illustrates movements of a subject's feet which wander from expected “Normal” or “Standard” foot spacing where the subject's left to right (Wander-1) spacing is larger than expected and right to left (Wander-2) spacing is shorter than expected. The unexpected movements would be determined by the mobility impairment algorithm to deviate from expected for either the “Normal” or “Standard” movement.

Further, a significant foot placement test while walking is to request the subject to walk toe-to-heal such that the subject places each foot at each step so that the heal of the front foot touches the toe of the back foot. This is a more difficult and perhaps stressful walking task for the subject and the mobility impairment assessment of the subject's movement can determine more subtle effects of and existence of concussion. Further, an even more difficult walking task is to request the subject to walk either regular walk or toe-to-heal walk but with the moving foot to cross over the stationary foot such that the subject's feet when both are stationary are crossed at every step in the walk. Mobility assessment of the subject, under the stress in this task, can determine even more subtle effects of and existence of concussion. Further, more stressful tasks such as having the subject running, such as on a treadmill, or riding a stationary bike, before requesting the subject to attempt any of the aforementioned tasks can precipitate the mobility assessment system to determine even finer subtle effects of and existence of concussion.

The above examples relate to an assessment performed in a controlled environment by a medical practitioner. The expert system may also be used in a normal non-clinical environment as a continuous, non-invasive risk assessment tool, such as but not limited to, a mobile computer and camera system implemented near an athletic playing field to provide quick on-sight assessment of athletes before, during or after play. Particularly if a player is suspected of having suffered a hit, shaking or injury to the head during play a prompt mobility assessment at the time of such occurrence could be critical in assessment for potential brain concussion and such that immediate action for medical attention can be taken as needed.

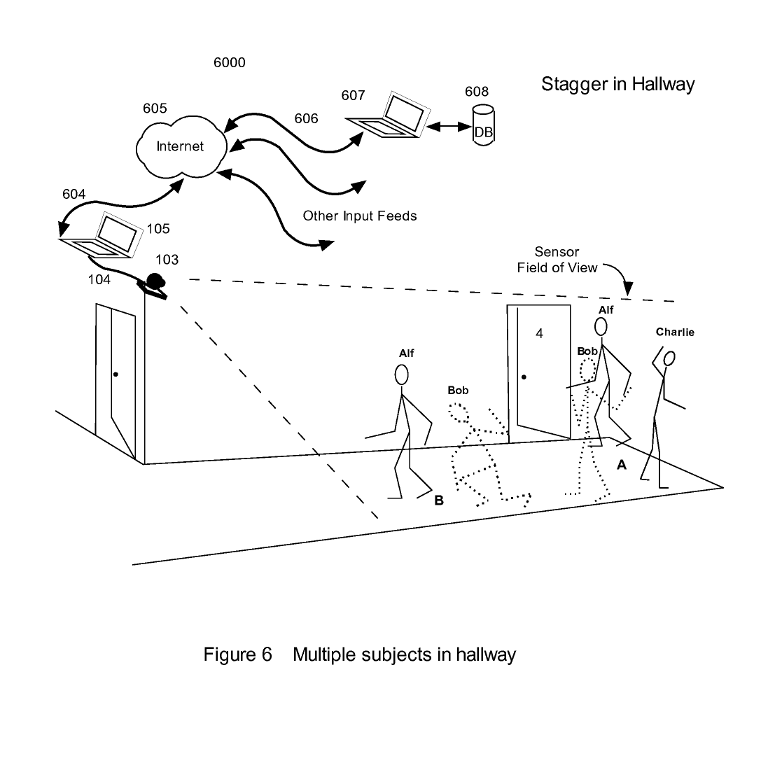

In a further example, shown in FIG. 6, a camera (103) is installed in a hallway and connected either wirelessly or through a cable (104) to a computer (105) implementing an expert system (603). That system may be located at the facility in which the observations are being conducted or may be connected through a network (604), such as the internet (605), connected through a network (606) to a remote facility (607) including databases (608). As shown in FIG. 6, illustrated are two subjects (Alf and Bob) walking to the left and one subject (Charlie) walking to the right in a hallway environment. In this example, say, Charlie waves to Alf and Bob and as Alf and Bob walk on further to the left, Bob attempts to wave back to Charlie but in so doing unexpectedly staggers. This is a non-clinical, everyday environment in which the system monitors the subject movement. Unexpected movements of Bob are determined by the mobility impairment algorithm administered to the video data of the system monitors to deviate from expected for either the “Normal” or “Standard” movement. The system can then alert the relevant parties of the potential for Bob to have brain concussion. This provides the capability of remote monitoring of a number of locations in both a real-time and recorded basis on a continuous basis with determination of potential concussion existence and associated risks for the observed subject. The central monitoring can therefore service a number of facilities and provide individual identification for future assessment or remedial action.

In each of the above examples, the assessment of a subject is performed using an expert system that administers a mobility impairment algorithm to determine appropriate action. The determinations from administering the algorithms to the data as performed by the expert system will vary according to the specific applications and the environment in which it operates. In each case however, administration of the mobility impairment recognition algorithms is used to determine the movement of the subject and to perform mobility assessment. That assessment may be assisted by reference to previous assessments where available and recommendations for mitigation and ongoing care may be determined by the expert system accessing a database of available options.

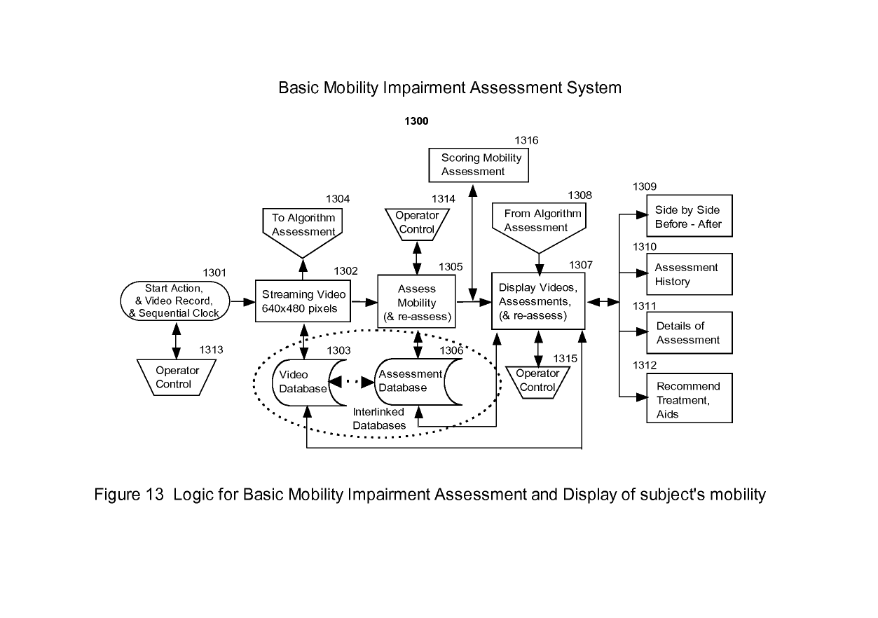

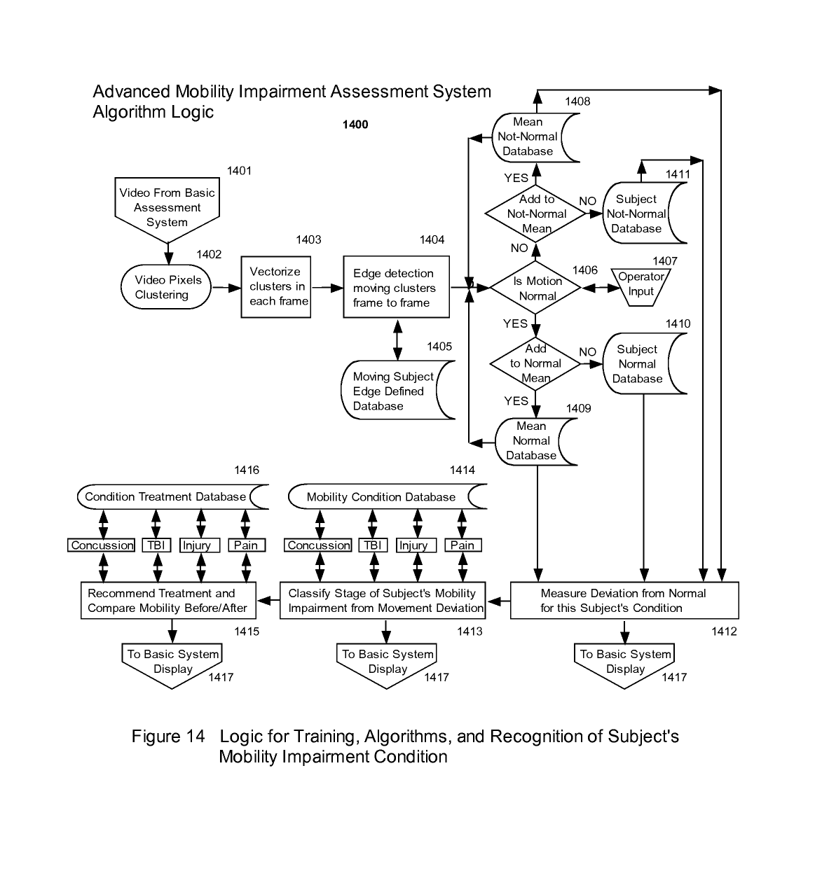

The implementation of the expert system can be considered as having two main linked components: a basic mobility assessment system (1300), as illustrated schematically in FIG. 13, and an advanced mobility assessment system (1400), as schematically illustrated in FIG. 14. The basic system (1300) permits an operator to control part or all of the assessment process and to input assessments of the mobility of the subject being assessed. The advanced system contains the algorithms and computer facility active logic engine neural networks decision computations with which the expert system determines the assessment outcomes and recommendations according to established parameters, the action assessment total score number, and the differential determination of current assessment to previous assessments, and generates reports of remedial actions, possible aids and healthcare procedures, to the subject, or to the subject's employers or to the caregivers of the subject.

As illustrated in FIG. 13, to perform an assessment, an input from the operator, indicated at (1313) starts the camera (1301) to generate a video stream and a clock stream, (1302). The operator can select from a menu the features to be activated and the mode of operation. The data are supplied to database collection (1303), and provides for output of that data stream to the advanced system (1304) for the computerized algorithm assessment decision determinations. A further operator input control (1314), permits assessment personnel to respond to prompts and assess (1305) the mobility of the subject, either from the real-time video data, or from previously captured data stored in video databases (1303). The assessment at step (1305) is performed by presenting prompts to the operator at (1314) which correspond to the inputs required for the expert system to determine the criteria to be scored in the standardized test that is applied. This step also allows the operator to use the system as a mobility impairment assessment tool by which the operator can introduce additional criteria from other databases including ones the operator has established. The aggregate scores are compiled by the scoring mobility engine (1316) and returned to the assessment database. The assessment is stored in a database (1306) and is linked to the corresponding video record in the database (1303). The data in the video database (1303) and results of the assessment in the assessment database (1306) can be presented and displayed and if desired the system can permit reassessment (1307) either by operator input control (1315) or automatically by the system.

Assessments derived from the advanced system algorithms (1308) are also integrated into the databases and display functions of the basic system (1307) if the operator (1315) has chosen to activate those functions. Additionally, the operator (1313) can decide and instruct the basic system which of the basic system assessments and the advanced system assessments are to be operating and storing data and assessments. This step also allows the operator to use the system as a mobility impairment assessment tool by which the operator can introduce additional criteria from other databases including ones the operator has established. The operator (1315) can instruct the basic system to display the data as raw video or, as discussed below as processed edge detected skeleton outline of the subject and to display these data and the resulting assessments of the mobility of the subject in a number of fashions. Typical modes of display include side-by-side video (1309) that depict the subject at different times, such as before and after treatment for the subject's physical or mental condition or disease; history of the subject's assessments over time (1310); details of any assessment and its components (1311); and any recommendations, treatments or aids (1312) that have been decided upon during the assessment process in either the basic system, such as by the operator (1315), or by the advanced system (1308). These displays can be video, numerical, charted, raw data, processed data, text, and audible, whether in electronic or non-electronic form and are generated by querying the data in the video database (1303) and assessment database (1306).

As illustrated in FIG. 14, the advanced system (1400) receives from the output (1304) of the basic system (1300) a video data stream (1401). The advanced system weight-averages and clusters the pixels (1402) in each video frame into groups, typically groups of 4 pixels by 4 pixels resulting in 19,200 such groups for each 640 by 480 pixel frame of video. Using known video processing techniques, the advanced system then detects the movement of each group from frame to frame by vector (1403) and based on the movement detects the edge of the subject by differentiating between those groups that are moving and those that are stationary on a frame to frame basis.

This may be performed by determining whether a given pixel with given color components M in image frame m moves or is displaced by 3 or more pixel spaces in any direction for this pixel in its location in the next image frame, n. If so then this pixel in frame n is identified as moved and assigned new color components, say green. If pixel M in frame m moved less than 3 spaces at its new location in frame n, then this pixel is identified as not moved and assigned new color components, say black. By computing the movement of all pixels from frame m to their locations in frame n and coloring green all those that move 3 or more spaces, and coloring black all those that move less than 3 spaces, a “ghost-like”, or skeleton motion-rendition of the subject's movements wherein all movement of the subject can be seen but details of the subject's face and identity are nearly impossible to recognize The skeleton images of the subject are far clearer, more detailed and easier to follow than a simple outline technique noted above as the 4 by 4 pixel clustering and edge detection resolved representation of the subject. However, in each case whether the edge detection or the skeleton techniques are used, the resulting image of the subject's movements protects the subject's privacy while the mobility impairment assessment and determination if brain injury or concussion is unimpeded.

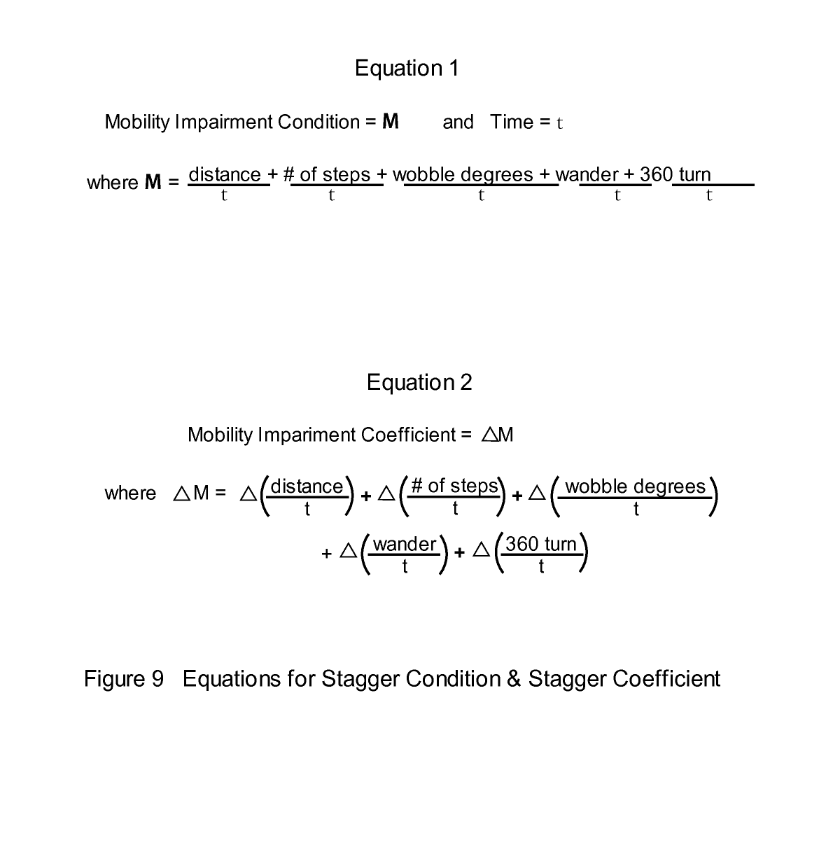

The edge detection (1404) creates an overall envelop outline of the object moving in the video referred to earlier as a skeleton outline which in this case is the subject being assessed, and stores that skeleton outline video motion in a database (1405). The motion is determined at (1405) by application of a mobility impairment algorithm to determine a mobility impairment condition M. A suitable mobility algorithm is shown as Equation 1 in FIG. 9 and for a given observation period t combines the distance travelled, the number of steps, the degree of wobble, the wander and the departure from the circular path of the 360 degree turn. It is preferred that the mobility impairment detection algorithms utilized are advances on and new derivations of those stagger algorithms of U.S. Pat. No. 7,988,647, and U.S. Pat. No. 7,999,857, and algorithms of U.S. patent application Ser. No. 11/011,973 and U.S. patent application Ser. No. 11/062,601, the contents of which are incorporated herein by reference. By using such techniques, it is possible to monitor if a particular movement indicative of a mobility impairment condition exists from determining the movements of a subject. Each of these evaluations may be made from the video data of the motion by determining the average deviation of a set of pixels representing the body, e.g. the average location of the centreline of the subject, to the normal path. The results are then combined to obtain the mobility impairment condition M. Of course the mobility impairment algorithm will vary depending upon the assessment being performed but in general provides a cumulative determination of deviation of movement from an expected path. Results can be determined for the subject being observed, in relationship to the subject's earlier assessment results, or assessment differential to a given starting assessment for the subject, or to a mean or average assessment assembled from many examples of normal mobility from assessment of many subjects, or to assessment assembled from many subjects at given stages of physical or mental condition such as brain concussion, TBI and Pain.

Depending on the test being performed, prior results of assessments may be loaded into databases (1408) and (1409) for relationship determinations. If the assessment is being determined in relationship to a prior assessment of the subject, then data regarding the subject are loaded into the databases. If however the assessment is being determined in relationship to a known condition or a particular class of subjects, data relating to that is loaded into databases (1408), (1409). The results in data bases (1408), (1409) are prior characterisations of movement as either “not normal” (1408) or “normal” (1409), and a determination of the mobility impairment condition M with those results enables the advanced system (1400) to decide if the motion of the skeleton outline data is normal. The stages of development of any such condition, or injury can be assessed by observing many subjects at given stages and assembling a mean “not normal” databases for a realistic representation of that stage of the condition, injury, brain concussion. Such mean “not normal” databases are often considered as training the mobility impairment algorithms to recognize such stages. Determination of the assessment differential of the assessment of the mobility of the subject to such mean “not normal” databases can provide a determination of the stage at which the subject's current condition, injury, or brain concussion exists and which permits the expert system to access its databases for determination of recommendations of treatments, aids or programs that might assist the subject to maintain or improve mobility and assist in the tracking and monitoring of rehabilitation from such injuries, concussions. Additionally, the expert system can compare past assessments before a specific treatment has been administered to the subject, to an assessment or assessments after the treatment has been administered from which the expert system can determine the change in mobility and effects of possible concussion from which the expert system can further determine the effectiveness of such treatment be it medication, physiotherapy, diet, psychological, surgery, healthy activity or simply the subject's personal healing process.

If there are no current data available with which the advanced system can make this decision, the operator (1407) can input this decision. In addition, by using this operator input, the advanced system can be trained to recognize and build databases of mean motions for either not-normal (1408) or normal (1409) motion of subjects to build databases categorizing these motions to, specific diseases, physical condition, mental condition, treatments, and the progress of any phases of these conditions. These mean databases that are “trained” to recognize these categories can pass this training to a mobility condition database (1414) and a condition treatment database (1416). The databases (1414) and (1416) are accessed through a deviation function (1412), that implements a further mobility impairment algorithm to determine a mobility impairment coefficient, AM, as shown in Equation 2 of FIG. 9. The mobility impairment coefficient is indicative of the deviation of the results either from a previous assessment of the subject or the mean or norm for assessments of similar subjects. The output of the deviation function is supplied at (1417) to the basic system as a factor to be included in the assessment. It is also supplied to a classification function (1413), and a recommendation function (1415) components of the advanced system (1400) for further evaluation. The mean can be determined for both “normal” movements and for “not normal” movements and these can be determined from assessments of many subjects thus deriving a general mean which can be arranged by age, sex, condition, illness, concussion, and the stages of same. The mean can also be determined for both “normal” movements and for “not normal” movements from assessments of the subject thus deriving stages of the condition, illness, injury, concussion and stages of same specific to the subject.

With sufficient mean data in the databases (1408) and 1409) the advanced system can determine, based on the determination of assessment differential with the mean data, the motion is normal (1410); or the motion is not-normal (1411) and can store the skeleton outline data of the subject accordingly. If appropriate, the operator (1407) can override the advanced system to input the decision that the subject is to be assessed by the system as either: normal and data stored (1410); or as not-normal and data stored (1411).

Having access to all the databases of the Mean Not-Normal (1408), of the Mean Normal (1409), of the Subject Not-Normal (1411) and of the Subject Normal (1410), the deviation function (1412) can determine the motions of the subject and can determine and assess the deviations from normal or from not-normal for the subject and provide (1417) these assessments to the basic system for database storage and display. The advanced system can then use these deviation determinations (1412) to classify (1413) the stage of the subject's mobility for the subject's condition, injury, concussion, treatments. By accessing the mobility condition database (1414) a determination of relationships with known classifications of mobility may be made, together with an assessment of the phase of the subject's concussion or condition. Assessment of mobility is an indicator in a number of mobility impairment conditions such as but not limited to injury, brain concussion, TBI, pain or illness. A determination of relationships with the data in the database (1414) for records relating to the same conditions provides an evaluation of the subject's condition which is provided (1417) to the basic system for database storage and display. The advanced system (1400) can then administer these classifications (1413) to query the condition treatment database (1416) and determine a recommendation of treatments, aids, actions for these concussions and conditions. The condition treatment database (1416) contains records of the specific treatments, aids and actions and provides (1417) these assessments to the Basic system for database storage and display.

It will be seen therefore that the incorporation of the mobility impairment algorithms into the expert system determining relationships in the records of prior assessments provides inputs to an individual assessment and determines suitable treatments and activities for the subject.

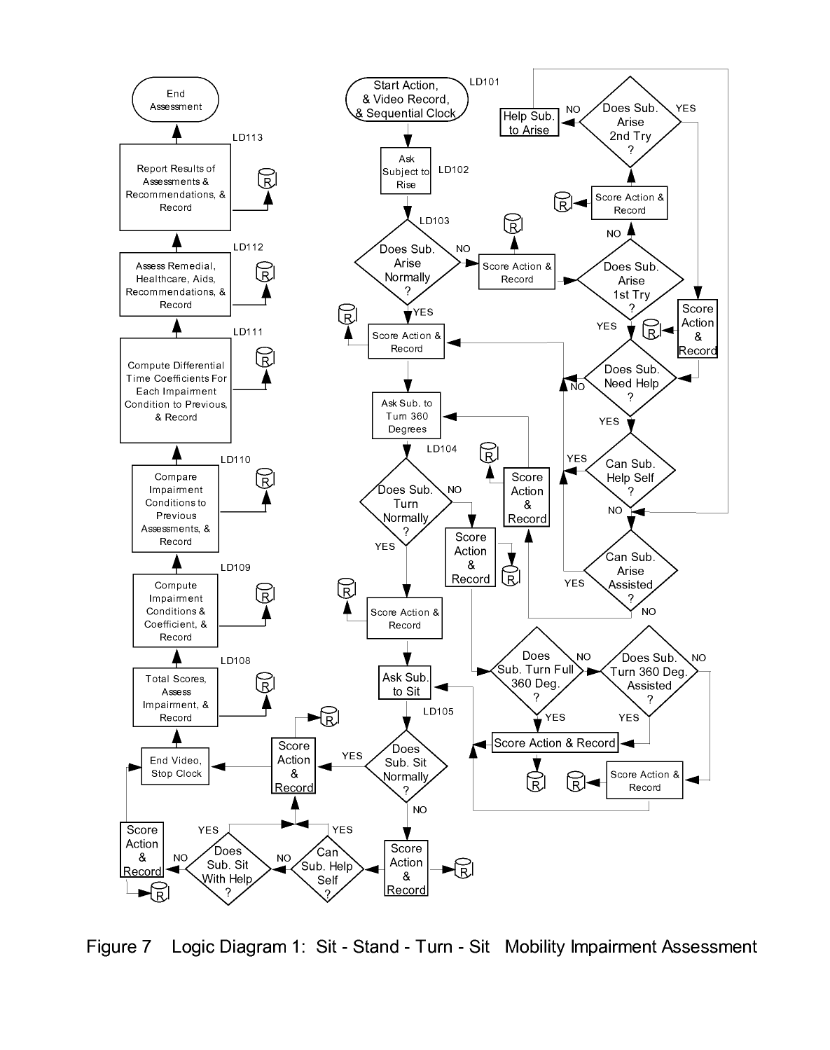

By way of example, the logic applied to a formal assessment under controlled conditions is illustrated in FIG. 7. FIG. 7 illustrates the sequence of events for the “arising from a chair” and “turning 360 degrees” test strategy shown schematically in FIG. 2. The assessor starts the assessment (LD101) and is prompted to ask the subject to rise (LD102). The data captured by the motion sensor is processed by administration of the stagger algorithm to determine if there are deviations from normal (LD 103). If no deviations are determined, the subject is assumed to have arisen normally and an appropriate score is accorded and recorded in the subject's record. If the subject arises normally, the assessor is prompted at (LD104) to ask the subject to turn 360°, and that motion is assessed by administration of the mobility impairment algorithm and scored accordingly. If a deviation is determined, the expert system accords an appropriate score which is recorded in the subject's record. The expert system then prompts the operator for further information and to perform further actions as the test proceeds. The relevant information is recorded at each stage to provide a cumulative score on the selected test. This is the functioning of the Basic System. During or upon completion of the test, the advanced system (1400) is invoked to determine through implementing its active logic engine neural networks decision engine to decide if the observed movement has been interpreted as a mobility impairment condition to which the system may assess the potential for concussion. These data are provided to the basic system (1300) for inclusion in the cumulative score. The system scores the actions of the subject's movements, totals all the scores and determines the mobility impairment of the subject (LD108), decides if mobility impairment conditions are detected, and computes the total mobility impairment as determined by the mobility impairment conditions and mobility impairment coefficient (LD109). The decision is subject to predetermined ranking of scoring. For example, give a maximum score of say, 100, at which the potential of concussion can be defined as: low for scores above 70; moderate for scores from 30 to 69; and high for scores from 0 to 29. During the training of the algorithms for “normal” and “not normal” these scoring rankings can be revised, developed and expanded as required

The use of the computer with an expert system capability to determine the mobility and potential for existence of concussion of subjects enhances the assessment of a subject. The expert system, by recording sequential time during the observations of subjects, can determine time intervals for subject's movements down to fractions of a second, say, one thousandth of a second and can measure and determine the subject's movements to such intervals. The time taken by the subject to make movements and the minute determination of these movements can be important data the expert system uses in its decision making processes. Further, even down to the image to image and pixel to pixel levels, the expert system can determine relationships of these timed movements from the subject's present assessment, to the timed movements of earlier assessments for the subject from which to detect change, deterioration, improvement in the assessment of risk of staggering or falling. The expert system can also determine relationships to “Standard” or “Normal” or “Personal” movements stored in its databases as part of the assessment.

Further, the movements of subjects being observed and algorithms administered by the expert system can be conducted in many different environments such as, but not limited to: testing environments like clinics, hospitals, practitioner's offices; or natural everyday surroundings like hallways, residences, apartments, walkways, streets, stores, malls; or athletic and sports activities playing fields, courts, gyms, professional stadiums; or confined spaces environments like industrial, commercial, experimental, and manufacturing. The observation and assessment of the mobility impairment or condition of mobility or condition of concussion are applications of the expert system. In some cases making these observations can influence or imply to the subject the need to perform and to do well on the assessment which can occur in a clinical environment. However, it is clear that these observations can be arranged to be unobtrusive, passive applications such as in natural everyday environments which can go unnoticed, and thus the observations do not affect the movements or performance of the subject.

In the above discussion of Logic Diagram 1, FIG. 7, “arising from a chair” and “turning 360 degrees” mobility risk of falling assessment examples, the expert system can use the advanced system (1400) to also determine relationships of the present assessment to previous assessments (LD110) and determine the differential times taken for each action using the sequential time clock (LD101). Determining the relationships of the times (LD111) taken for each action in a previous assessment to the present assessment, the expert system can determine the differential time coefficients for each mobility impairment condition or action from which to further determine if the condition or action has remained the same, improved or deteriorated. The system can also determine the relationships of the mobility impairment condition (Equation 1) from which a mobility impairment coefficient (Equation 2), as illustrated in FIG. 9, can be used to further determine the mobility impairment condition and changes in that condition with time. This relationship is in addition to the relationships and decisions of mobility described in (1400) and can further refine the determination of the mobility assessment, and stage of the subject's concussion, illness, pain, or disease, and treatment effectiveness, and progress in same.

From the above computer facility active logic engine neural networks decision administrations, the expert system determines (LD112) the assessment outcomes and recommendations according to established parameters, action assessment total score number, and differential relationships of current assessment to previous assessments, and determines (LD113) remedial actions, possible aids and healthcare procedures for the subject, their family, their employer, their health providers and caregivers of the subject. These recommendations could be, but are not limited to, assigning a repeat of the assessment for confirmation, assigning a follow up assessment upon confirmation of mobility impairment, and reporting electronically or by hardcopy output to the health and caregivers or the subject's family or the subject's professional advisors or the subject's employer.

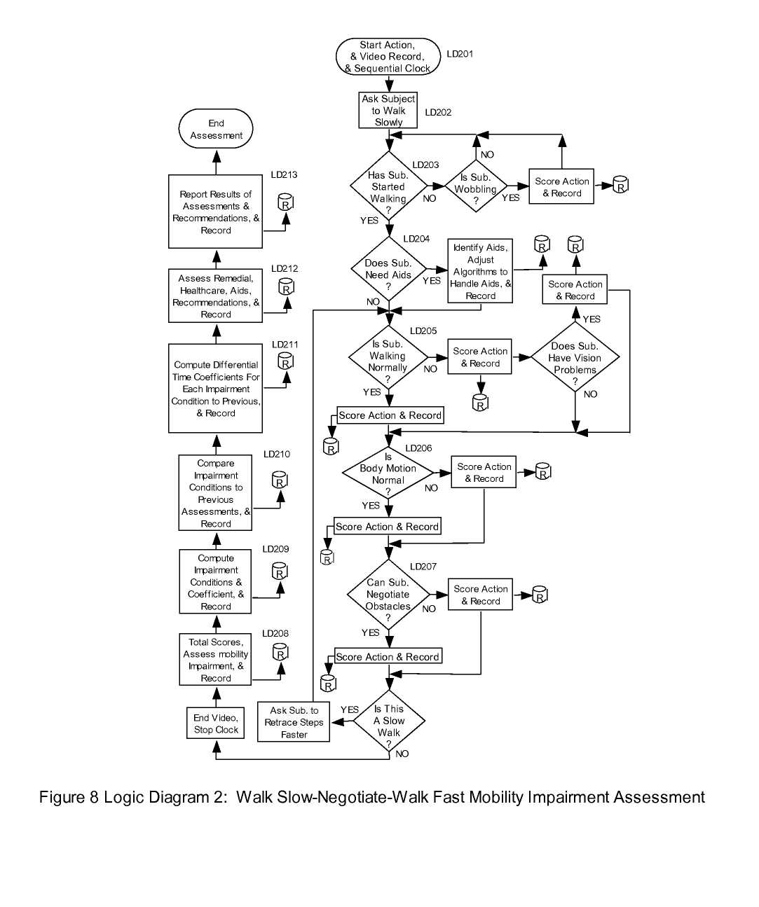

FIG. 8, Logic Diagram 2, Walk Slow-Negotiate-Walk Fast Mobility illustrates another test of the subject's mobility, mobility impairment and potential existence of concussion, illness, pain or disease, similar to that of Logic Diagram 1. In the Walk Slow-Negotiate-Walk Fast Mobility, the assessor starts the assessment (LD201) and the subject is asked to walk slowly (LD202), perhaps showing hesitation (LD203) or needing aids (LD204) to walk normally (LD205) without body sway (LD206), to negotiate obstacles (LD207) and to retrace this path at a faster pace. The expert system, observing and video-recording, and administering the algorithms (LD203-207), while operating in real time during the live observation process or operating off-line administering the algorithms to the recorded video following the observations video-recording the movement, for which the system employs computer facility active logic engine neural networks decision determinations in a computer as administered to the video data of those movements according to specific algorithms and tests, scores the actions of the subject's movements, assesses the mobility of the subject, and determines the total risk of falling in relationship to the determination by this assessment (LD208). The system further determines if the observed movement has been interpreted as a mobility impairment condition to which the system may further determine the potential for existence of concussion, illness, pain or disease and determines if mobility impairment conditions are detected, and computes the total risk for existence of concussion, illness, pain or disease, as determined by the mobility impairment conditions and mobility impairment coefficient (LD209).

In the above “Walk Slow-Negotiate-Walk Fast Mobility” risk of falling assessment examples, the expert system can also determine relationships of the present assessment to previous assessments (LD210) and determine the differential times taken for each action using the sequential time clock (LD201). Relating the times (LD211) taken for each action in a previous assessment to the present assessment, the expert system can determine relationships of the differential time coefficients for each mobility impairment condition or action from which to further determine if the condition or action has remained the same, improved or deteriorated. The system can also determine the mobility impairment condition (Equation 1) from which a mobility impairment coefficient (Equation 2), as represented in FIG. 9, can be used to further determine the mobility impairment condition and changes in that condition with time. This determination is in addition to the determinations and decisions of mobility described in (1400) and can further refine the assessment of mobility, and stage of the subject's condition, concussion, illness, pain or disease, and treatment effectiveness, and progress in same.

From the above administrations of computer facility active logic engine neural networks decision determinations, the expert system determines (LD212) the assessment outcomes and recommendations according to established parameters, action assessment total score number, and differentials of current assessment with previous assessments, and reports (LD213) remedial actions, possible aids and healthcare procedures to the caregivers of the subject.

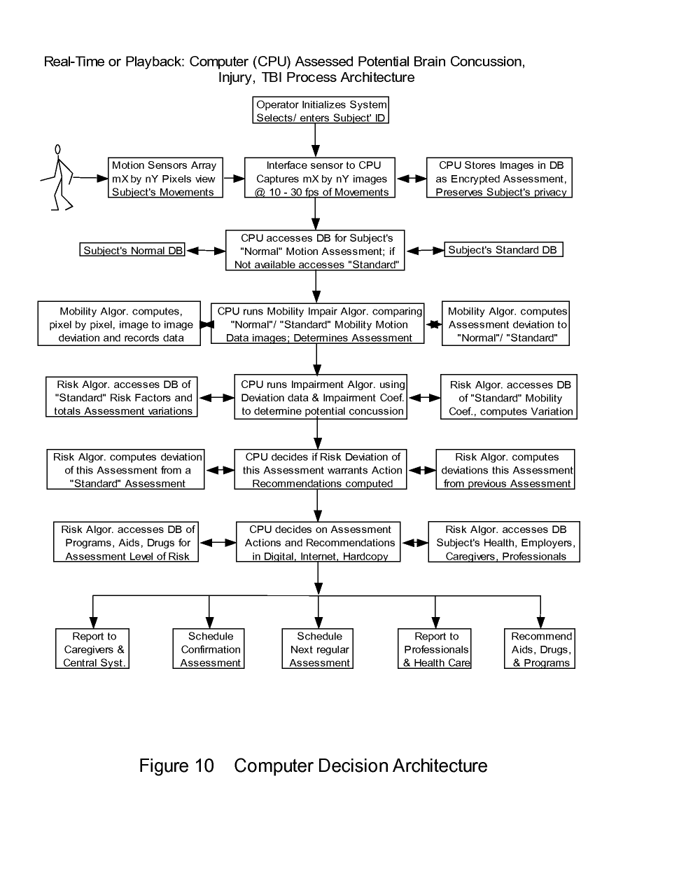

The advanced system (1400) includes the administration of the mobility impairment condition M and the mobility impairment coefficient as components utilized in the assessment of the potential existence of concussion, illness, pain or disease. FIG. 9 illustrates the format of these determinations for Mobility Impairment Condition (Equation 1) and the Mobility Impairment Coefficient (Equation 2) as performed by administration of the computer active logic engine algorithms as part of the continued observation of the data. The process architecture for the risk of falling assessment determinations by the expert system are determinations derived from decisions made from administration of the algorithms to video data of the subject as illustrated in the block diagram of FIG. 10.

In FIG. 10, the process architecture for the expert system assessment of the potential existence of concussion, illness, pain or disease, is illustrated in block diagram format. The process begins as an operator initializes the expert system which begins observations and recordings of the motions of a subject, capturing images at 10-30 frames per second with timing markings of 1/1000 sec. The recordings can be encrypted for security and privacy. The expert system can assess the observations of the subject's motions, in real-time or after recording them, in determinations of relationships to earlier “Personal” observations of the movement of the subject or to “Standard” observations of similar subjects as stored in the systems databases. Determination of the relationships of the present observations to the “Personal” and “Standard” base-line movement data have been explained earlier, and are used by the expert system running the Mobility Impairment Algorithms to determine the potential existence of concussion, illness, pain or disease Assessment. Using the timing markings the system can determine the deviations from “Personal” or “Standard” by image to image and by pixel to pixel to determine Mobility Impairment conditions and the Mobility Impairment Coefficients related to the potential existence of concussion, illness, pain or disease. Thereafter, the results for that particular assessment may be determined as in relationship to the databases to obtain the change for that subject from previous assessments and/or the status of that subject relative to norms in particular categories.

FIG. 10 illustrates that depending upon results of these factors determined by the expert system, the system then can, as explained with respect to FIG. 14, determine relationships in the databases and consider other information about the subject, such as but not limited to, use of drugs, health and condition, use of mobility aids, and previous data from caregivers and professionals, which together with the current assessment results, the expert system can determine actions to follow, recommendations and the completed current assessment. The expert system can then determine scheduling of further assessments, such as but not limited to confirmation or regular assessments, and can determine relationships with databases of recommendations related to the current assessment results with which the system can make decisions as to use of potential mobility aids, drug regimes, and programs, such as but not limited to, exercises or physiotherapy, and to report these results and recommendations to caregivers, professionals and health care groups, family, employers as well as to other centralized data systems for recording and further.

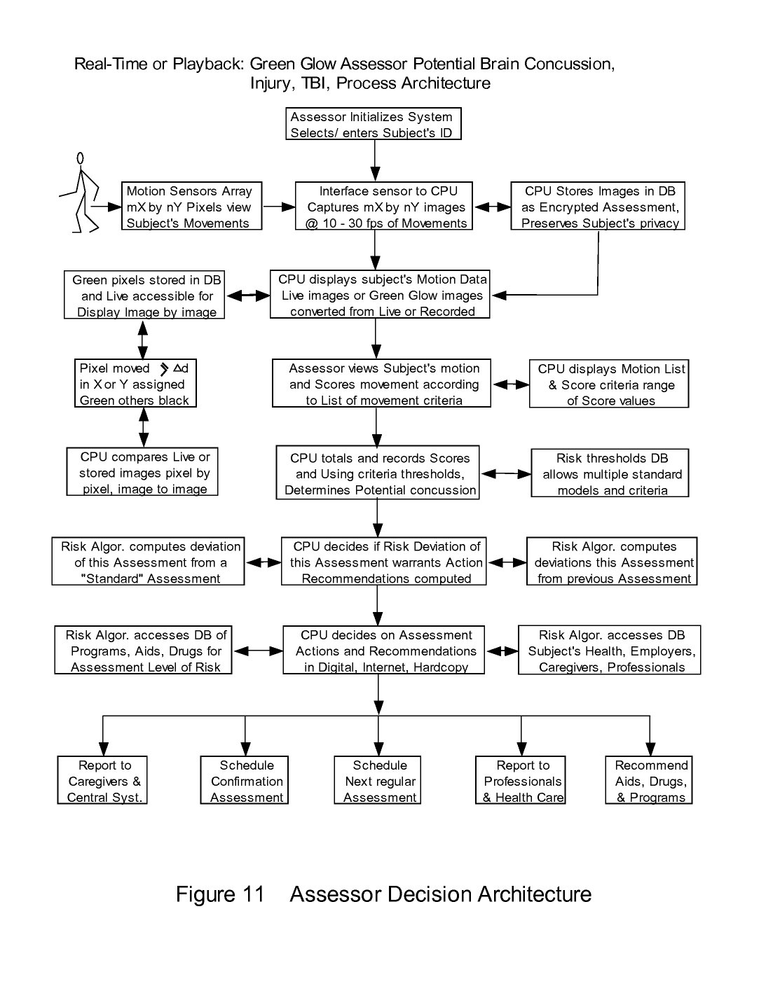

FIG. 11 illustrates the case where the capability of the expert system to present the “green glow” imagery rather that the video data imagery has been chosen. In a further preferred embodiment of method and apparatus of the invention, a qualified personal Assessor can make the decisions from live real-time or recorded playback utilizing computer assistance. In FIG. 11, a block diagram illustrates the process architecture for a human Assessor to determine the potential existence of concussion, illness, pain or disease, by observing the movement of a subject being observed and recorded by the expert system as described earlier and shown in FIG. 10. In the FIG. 11 case, however, the Assessor is only being assisted by the expert system which can display the observations live or in playback and in video movements or the “Green Glow” movements. The expert system can display for the Assessor, lists of accepted movement criteria and permit the Assessor to select and score the observed subject's movements, and the expert system can record these selections and scores. At this point the Assessor can decide to use only these scores and to have the expert system compute total scores and determine the risk of falling according to the established criteria. The Assessor can then determine what results and recommendation to make and whom to report them for follow up actions.

Further however, once the Assessor has completed the assessment of the movements of the subject, the Assessor can have the expert system proceed as earlier described for FIG. 10, to determine the variations, deviations, mobility impairment conditions and coefficients, and combine these results with the Assessors determination of the potential existence of concussion, illness, pain or disease, for the system to then administer the active logic engine algorithms and arrive at a new determination of the potential existence of concussion. By combining the Assessor's determinations and the expert system's determinations, the resulting assessment of potential existence of concussion, illness, pain or disease, may be improved. The Assessor can then have the expert system determine what results and recommendations to make and to whom to report them for follow up actions as earlier discussed and illustrated in FIG. 10.

Additionally, the expert system could decide the potential existence of concussion, illness, pain or disease, is sufficiently great to recommend installation of facility for a 24-hour video/motion monitoring/recording system in the subject's living quarters or where the subject is known to move about. Such a system could be arranged to erase the previous 24 hours of recording if the system has been notified by the subject's caregivers to do so. If saved, this recording could be used for further determinations of the occurrence of a stumble, stagger or fall and, could provide information for subsequent response of authorities. A more advanced installation of a facility could be recommended to include with the 24-hour system an additional computerized movement mobility impairment such as indicated herein, with which the facility could automatically detect a mobility impairment condition which would tell the facility to retain part or all of the 24-hour video/motion monitoring/recording and to start a new 24-hour video/motion monitoring/recording. And yet a more advanced installation of a facility could be recommended to include with the 24-hour system, additional computerized movement mobility impairment determinations, and a fall detection capability. Such a more advanced facility could not only automatically determine a mobility impairment condition but it could also determine a potential existence of concussion, illness, pain or disease, for which notification of the condition to the subject's health professionals, family, employers and caregivers could provide quicker response and assistance being given.

All three of the above facilities could utilize data encryption technology for protection of privacy, but with legal authority could be viewed to establish what movement occurred, where it occurred, and possibly why it occurred. This information, accompanied by the assessment results, could provide valuable assistance to improve the care given to the subject, improve the quality of life for the subject and provide important evidence in case of any legal, insurance, liability, or publicity actions that could arise from the mobility or lack of mobility of the subject.

Privacy can also be a requirement for the video recording used in the fall prevention and mobility assessment methods and apparatus being revealed in this patent. Several different methods can be used to render the subject not recognizable in the assessment video recordings of the subject. Methods can include electronically altering the subject's facial features in the video recording, removing color components in the video recording, and electronically erasing the head of the subject in the video recording.

In a preferred embodiment of the apparatus and methods of this invention, the video processing of a skeleton image can transform the images of the subject in the video recording to become an outline of the subject with full retention of all movements of all of the subject's body including feet, legs, trunk, arms, hands and head while rendering the recording devoid of the information needed to identify the subject. In this way the subject's privacy can be maintained while the mobility fall risk and mobility assessment is unimpeded.

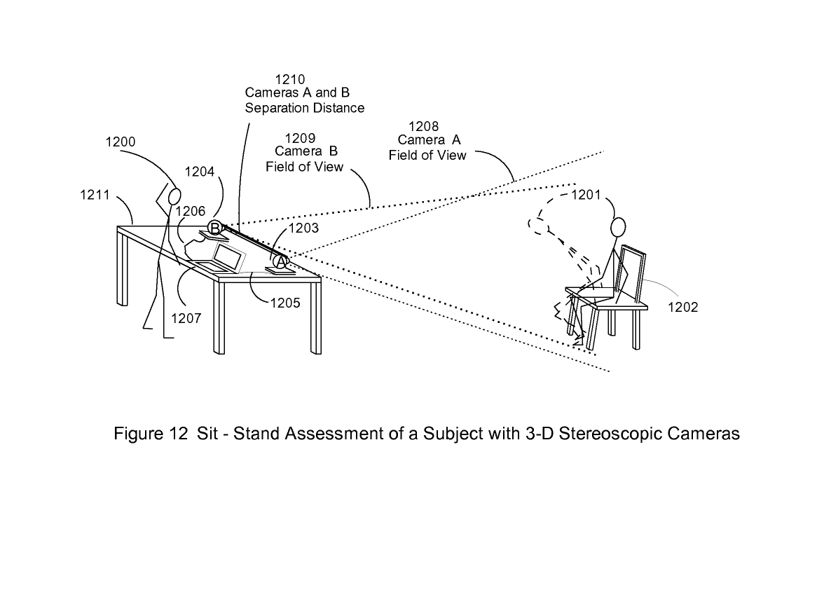

In an alternative embodiment images from multiple cameras may be used as shown schematically in FIG. 12 (camera A 1203 and camera B 1204) sitting on a table (1211) or any other stand or facility. The cameras are separated by a distance (1210) and observe the subject with separate fields of view (camera A view 1208 and camera B view 1209). The video data from each of the cameras is connected via cables (1205) and (1206) or by wireless connection, to the controlling and data collecting computer facility (1207) of the expert system as operated by the test facilitator (1200). One of these cameras could be an infrared illumination source and receiving detector and the other could be a visible detector such as but not limited to the Microsoft Kinect duel camera system utilized in the Microsoft games console. The use of the Kinect in this Mobility Assessment system has been found to be an inexpensive 2-camera sensor system with the added advantage of significantly improving separation of the background from the moving image of the subject. The data are composed into a stereoscopic 3-dimensional (3-D) representation of the subject's movements using known image reconstruction techniques, and can transform the images of the subject in the video recording to become an outline of the subject with full retention of all movements of all of the subject's body including feet, legs, trunk, arms, hands and head while rendering the recording devoid of the information needed to identify the subject. In this way stereoscopic 3-D modelling of the subject's movement can provide more precise and more accurate determination the subject's movements and the subject's privacy can be maintained while the mobility and mobility impairment assessment and measure of potential existence of concussion, illness, pain or disease, is unimpeded.

Using the methods and systems described above to observe and video record the movements of subjects, using a wide variety of tests and algorithms employing computer facility active logic engine neural networks decision computations, it is possible to determine the mobility impairment and with appropriate detection facilities determine the potential existence of concussion, illness, pain or disease. The results of these assessments and computations can be used by the expert system to determine and recommend particular mobility aids such as use of canes, walkers and wheel chairs and implementation of remedial programs such as physiotherapy, exercise and strengthening routines, subject's training and relearning brain functions and capabilities, as well as healthcare programs, any or all of which can be preventative actions for problems of potential existence of concussion, illness, pain or disease, as determined from these assessments. Reporting of these assessments and actions, whether electronically, such as computer to computer or e-mail, and digitally such as magnetic media such as CD's, DVD's and hard copy printed and graphic documentation, provided to the assessed subject's' caregivers, professional advisors, family members, employers or the subjects, can be vital in informing them of potential existence of concussion, illness, pain or disease, and planning continued mobility impairment assessment detection and response, with the intention of predicting and preventing further such concussion; and for illness, pain or disease, curing, arresting or reversing effects of the illness, pain or disease. The reporting of these assessments is vital in discovering the potential existence of concussion and additionally it is vital for preventing, predicting and planning to manage further concussion as well as and for illness, pain or disease; curing, arresting or reversing effects of the illness, pain or disease.

In clinical tests conducted to date to test and validate the assessment methods and apparatus it was found that the methods and apparatus were well received, functional and highly accepted as providing valuable information. The linkage relationships determined between current and previous assessments in evaluating the changes in mobility and mobility impairment and potential existence of concussion as well as and for illness, pain or disease curing, arresting or reversing effects of the illness, pain or disease was also recognized to be effective.

The system described above has the capability to determine relationships of a subject's present assessments to the subjects previous assessments whereby the expert system can determine and measure the changes in any of the actions and motions of the subject specifically tailored to the subject's individual conditions and health. The expert system not only has databases of information on what are considered normal movements and actions of persons depending on age, sex, health condition and drug use, but also has similar databases specific to the subject being assessed, and thus the expert system can also base-line calibrate its decision-making determinations to what are considered normal movements and actions of the subject being assessed. Determining the relationships to the subject's base-line the expert system can further determine if the present assessment is normal or if it indicates a mobility impairment condition and possible potential existence of concussion, illness, pain or disease. If the system determines that a mobility impairment condition exists, then the system can determine relationships of the present assessment to previous assessments for this subject to further determine changes in the mobility impairment conditions. Further, if video monitoring in areas where the subject moves about, such as but not limited to, in a residence, home, hospital, playing and sports fields, professional stadium and sports entertainment facilities or natural environments are implemented as the earlier discussion noted, the expert system can determine relationships of these data with which the system can determine the mobility impairment and changes in the subject's mobility in the subject's daily living environment from which the system can determine more comprehensive preventative and remedial practices, health and well-being programs, mobility aids, and monitoring programs for improved quality of life activities, work related activities, monitoring of rehabilitation programs and their success or failure or modifications specific for the subject.

In either real-time or post-recording, the expert system can be the decision-making facility which permits the actual operation of the system and assessment to be done by regular staff of the subject's employer, or clinic, or athletic or sports facilities without the need for highly qualified and expensive professional personnel. The expert system can also utilize its determinations to review part or all of the assessments done for the subject so as to provide the history of the subject's mobility, impairment, potential existence of concussion, from which the system can determine further risk of concussion, illness, pain or disease, and remedial practices and recommendations based on the full history of the subject's mobility as stored in the system's databases and, if access is available to the expert system, by making these determinations with data from databases of other systems.

The apparatus and methods described above can also allow authorized personnel, such as professional physiotherapists, neurologists and concussion specialists to review the data and the determinations made by the system, and the system can allow that personnel to score new data values for any and/or all mobility observations of an assessment thereby creating a new or updated assessment which can be recorded accordingly.

The expert system can also transmit the determinations and recommendations of the current as well as previous assessments via electronic, digital, analogue or hard copy media to the subject or the subject's caregivers, medical practitioners, family, employers or legal representatives. Results so transmitted can allow others to review the assessments and data allowing them to provide second opinions and guidance for the subject. The expert system utilizing standard plotting methods can provide viewing of these determinations of assessments and results over selectable time periods, including the entire time that assessments have been done for the subject, thereby allowing viewers improved ability to follow and understand the changes in the subject's mobility. This historical review of assessments can permit improved recommendations to be made by those others provided access such as, but not limited to, scheduling further assessments, ordering mobility aids, planning new activities and body-strengthening routines and implementing video monitoring activities, as preventative methods for elimination of any further concussion, and their detection measures that could be implemented.

As noted above, the system may use 2-D video data or from three dimensional, 3-D video data. For many motions such as staggering or wandering in the walking path of subjects, 2-D video data may be adequate to determine and assess the mobility of the subject. However, for some motions such as the quick motion of uncontrolled shaking of the hands or head of subjects, 3-D video data may be required for assessing the mobility of the subject. Additionally, use of 3-D in which one camera is an infrared illumination source and receiving sensor and the other is a color visible camera can greatly improve the separation of the moving subject being assessed from the background environment in the viewing scene resulting in significantly improved mobility assessments.

From the above it will be clear the assessment methods and apparatus described could be applied to many environments, such as, but not limited to, hospitals, private homes, hotels, commercial establishments, doctor's offices, clinics, drugstores, mobility-aids stores, and in the broad sense anywhere people are moving about such as sports and athletic facilities, playing fields, gyms, employment facilities. Also it will be clear to anyone versed in the healthcare field that many different algorithms, test parameters, action scoring methods and determinations can be implemented, including, but not limited to, mobility impairment algorithms, time derivative determinations and mobility testing, such as those we reveal as incorporated into the computer facility active logic engine neural networks decision determinations methods and apparatus with which we can assess mobility impairment and potential existence of concussion, illness, pain or disease, the preventative outcomes and recommendations to reduce further mobility impairment and potential further concussion, and for improved quality of life for assessed subjects. Further, it will also be clear that the methods and apparatus, assessments and recommendations facilitated by the expert system can have application to any subject persons regardless of their age, health, sex, location or activity. Also, it will also be clear that the methods and apparatus, assessments and recommendations facilitated by the expert system can have application to assessment of and the progression of concussion, and the effects of treatments and rehabilitation regimes whether trials or long-term such as but not limited to drugs, physiotherapy, nutrition, exercise, and success or failure of those treatments, for those diseases, illnesses, pains such that applications are not limited to only those concussions, illnesses, pains or conditions disclosed herein. The systems and methods of detecting brain concussion, illness, pain or disease, by determining mobility in this example have been successfully applied to the mobility impairment assessments of concussion, illness, disease, and pain of spinal injury subjects including the follow up tracking of their rehabilitation and healing of the mobility impairments over time.

From the above, it will be clear the determination of mobility impairment will include the deterioration of the walking gait of a subject. It has been shown by extensive studies that the deterioration in mobility, including gait, of a subject has been directly correlated to neurological deterioration of the subject. Dr. Dean M. Wingerchuk at the Mayo Clinic in Rochester Minn. has reported “Gait analysis adds objective, reliable outcome measures sensitive to detecting neurological deterioration.” Dr. Wingerchuk states that “Gradual deterioration in ambulatory function is one of the major manifestations of progressive forms of Multiple Sclerosis”. At the Alzheimer's Association International Conference 2012 in Vancouver, Canada, three independent research studies each surveying more than 1,000 people, all confirmed mobility deterioration in gait of subjects directly reflected their neurological deterioration due to their Alzheimer's dementia. The studies were conducted by Dr. Stephanie A. Bridgenbaugh of the Basel Mobility Center in Basel, Switzerland; Dr. Mohammad Ikram at Erasmus MC Rotterdam, the Netherlands; and Dr. Rodolfo Savica of the Mayo Clinic Study of Aging, Rochester Minn.

From the above, it will be clear the assessment methods and system means described could be applied to the determination of mobility impairment including the deterioration of the walking gait of a subject to determine the potential existence of brain related illnesses including but not limited to Multiple Sclerosis and Alzheimer's dementia.

From the above it will be clear the assessment methods and system means described could be applied to animal subjects. Animals can't talk and tell us what ails them, so the non-invasive, objective, computerized mobility assessment system, of the methods and apparatus described could provide a useful tool in diagnosing health problems in animals including but not limited to concussion. An example of this would be in the case of Mad Cow Disease in which brain damage in its early stage causes mild walking difficulty for the cow, stages advancing to stumbling, then to inability to walk and finally to death of the cow. Deriving the normal and not-normal mobility assessment databases of the system for movement of animals such as but not limited to cows, could permit the assessment methods and apparatus described to detect the effects of and existence of this deadly brain deterioration condition which if not recognized early enough in this example often results in the destruction of entire herds fearing spread of Mad Cow. Further, mobility assessment of animals including but not limited to those kept as pets, could be used to assist veterinarians, owners and caregivers of these animals and pets, to provide better monitoring of the health and wellbeing of the animals and pets, including but not limited to detecting the effects of and existence of concussion. Further, it will be clear that these assessment methods and system means may be applicable to humans contracting the possible Creutzfeldt-Jakob neurodegenerative brain disease related to Mad Cow Disease.

In this example, the expert system administers the active logic engine algorithms to the data available to identify that a mobility impairment condition exists in one or more movements in the current assessment and accesses a data base to determine relationships of this mobility impairment condition to a previous assessment for this subject, stored in the database component of this system, to determine if this mobility impairment condition was detected in a previous assessment. If the mobility impairment condition did so exist, the computer system, administering time derivative determinations, calculates the rate of change in the mobility impairment condition between successive assessments for this subject. The computer facility, using a predetermined baseline matrix of outcomes, then determines if a critical mobility impairment condition exists and, comparing to previous assessments, determines if a deterioration in the mobility impairment condition has occurred, and if so occurring computes the rate of change of this deterioration. This active logic engine algorithm function of the computer system can apply equally to the concussions and conditions of the subject as discussed herein.

What we claim is:

1. A system for determining the mobility and mobility impairment of a subject, said systems comprising a motion sensor or sensors to observe movement of a subject and determine a data stream representative of such movement, an active logic engine administered to determine abnormalities and impairments in such motions and determine similarities of said abnormalities or impairments to at least one known norm and an allocator operable in said active logic engine to determine whether said abnormalities or impairments are within said known norm and a means for recording said determinations.

2. A system according to claim 1 including a pair of databases, each of which provides a respective known norm determined from the contents of the database, said active logic engine allocator administered to determine said abnormalities or impairments to said known norms provided by said database to determine relationships of the said abnormalities or impairments to said norms.

3. A system according to claim 2 wherein said active logic engine is operably administered to add said determined abnormalities or impairments to one of said databases.

4. A system according to claim 3 wherein said active logic engine administers mobility or mobility impairment algorithms to determine abnormalities or impairments.

5. A system according to claim 4 wherein said active logic engine incorporates an active logic decision engine to administer said mobility or mobility impairment algorithms.

6. A system according to claim 2 including a mobility and mobility impairment condition database containing records of abnormalities and impairments of prior assessment determinations to permit the said determined relationships to be administered to prior assessments for determining further relationship outputs.

7. A system according to claim 6 wherein records of said mobility and mobility impairment condition database include categories of condition to permit said outputs to be administered for determination of assessment differential to selected conditions and a determination of a classification of said condition determined.

8. A system according to claim 7 including a treatment database to permit administration of said classification to determine a treatment regime as a cure remedial action for said condition.

9. A method of determining mobility and mobility impairment of a subject comprising the steps of recording motion of said subject, determining condition of said subject for abnormalities and impairments of such movement, determining relationships of said abnormalities and impairments to known norms and determining whether said abnormalities and impairments are within a known norm.

10. A method according to claim 9 wherein said abnormalities and impairments relationships are determined administering a mobility and mobility impairment assessment algorithm.

11. A method according to claim 10 including administering the step of generating said known norms from a database of prior algorithm determined relationships.

12. A method according to claim 11 including the step of administering algorithm determined relationships of said abnormalities and impairments to prior records of said subject.

13. A method according to claim 11 wherein said abnormalities are administered for determination of relationships to prior assessments of different conditions.

14. A method according to claim 1 where said active logic engine incorporates administration of active logic engine algorithms for the purpose of determination of video data of a subject's movement, using said allocator administration of mathematical algorithms permitting determinations of relationships and assessment of deviations from calibrated “standard” determinations of “normal” mobility of healthy subjects to determine the mobility impairment potential for existence of brain concussion of said subject.

15. A system according to claim 8 were said cure is for brain concussion.

16. A method according to claim 14 where said administration of said active logic engine can determine the mobility impairment potential for existence of brain injury of said subject.

17. A system according to claim 8 were said cure is for brain injury.

18. A method according to claim 14 where said administrations of active logic engine algorithms can determine, frame by frame, the video of the movement of said subject contained in the said video data by administering isolating determinations of the subject from the background and administering a set of control points on the image that describe the movement and administering a grid segmentation on the image with which the said administrations of said active logic engine algorithms can determine electronic or mathematical and matrix determined signatures in the time domain that describe the mobility impairment as a determination of brain concussion of said subject being viewed and can record said determined signatures in databases.

19. A method according to claim 14 where said administration of said active logic engine can determine the mobility impairment including the deterioration of the walking gait of a subject to determine the potential existence of brain related illness including but not limited to Multiple Sclerosis and Alzheimer's dementia.

20. A method according to claim 14 where said administrations of active logic engine algorithms can be administered to said prior records of said conditions to determine standard calibrated information defining impaired mobility of subjects due to concussion influences on the body for administering determination of assessment differential to real time or recorded information determined from subsequent said observed video data of a subject.

21. A system according to claim 2 wherein said active logic engine is administered to add said determined abnormalities or impairments to one of said databases where said abnormalities or impairments are determined as calibrated “standard” determinations of “stages of impairment” for subjects at a given stage of a given brain related concussion or injury and stores said stages in said databases.

22. A system according to claim 2 wherein said abnormalities or impairments are said determined for said calibrated “standard” determinations of said “stages of impairment” for subjects wherein said abnormalities or impairments are disabilities of said subjects due to, such as but not limited to, diseases, injuries, accidents, such as but not limited to, work related activities, recreational activities, domestic activities, including but not limited to activities that activate or reactivate the subject's present or prior physical or mental conditions, abnormalities or impairments.
